(19) United States
(12) Patent Application Publication
(10) Pub. No.: US 2023/0250200 A1
(43) Pub. Date: Aug. 10, 2023

(54) IMPROVED CATALYSTS FOR INTERMOLECULAR CYCLOADDITION

(71) Applicants: EXXON MOBIL CHEMICAL PATENTS INC., BAYTOWN, TX (US); THE TRUSTEES OF PRINCETON UNIVERSITY, PRINCETON, NJ (US)

(72) Inventors: Irene C. Cai, Webster, TX (US); Gursu Culcu, Humble, TX (US); Tzu-Pin Lin, Seabrook, TX (US); Jo Ann M. Canich, Houston, TX (US); Frank N. Raushel, Baytown, TX (US); Alex E. Carpenter, Austin, TX (US); Hua Zhou, Missouri City, TX (US); Danielle G. Singleton, Houston, TX (US); Paul J. Chirik, Princeton, NJ (US); Megan Mohadjer Beromi, Pasadena, MD (US); Carli Kovel, Princeton, NJ (US)

(73) Assignees: EXXONMOBIL CHEMICAL PATENTS INC., BAYTOWN, TX (US); THE TRUSTEES OF PRINCETON UNIVERSITY, PRINCETON, NJ (US)

(21) Appl. No.: 18/156,947

(22) Filed: Jan. 19, 2023

Related U.S. Application Data
(60) Provisional application No. 63/307,738, filed on Feb. 8, 2022.

(51) Int. Cl. C08F 4/80; C08F 2/60; C08F 110/14; C08F 136/06

(52) U.S. Cl. CPC C08F 4/80; C08F 2/60; C08F 110/14; C08F 136/06

(57) ABSTRACT

The present disclosure relates to iron-containing compounds including a 2,6-diimino(heteroaryl) ligand useful for producing substituted-cyclo-alkanes, such as vinyl cyclobutanes. The present disclosure provides new and improved iron-containing catalysts with enhanced solubility in hydrophobic (nonpolar) solvents.

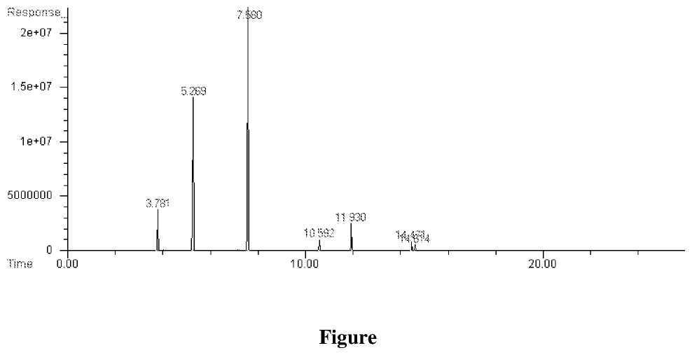

### FIELD

The present disclosure relates to 2,6-bis(imino)pyridyl iron compounds to make substituted cyclobutanes.

### BACKGROUND

The [2π+2π] cycloaddition of two olefins results in the formation of a strained cyclobutane ring. Although symmetry forbidden under thermal conditions, this transformation can be accomplished using transition metal-containing catalysts. For example, iron-containing catalysts have been used to make alkene-substituted-cyclobutanes, such as vinylcyclobutane, however the solubility of the iron-containing catalysts impacts the ability to obtain alkene-substituted-cyclobutanes, such as vinylcyclobutane, at good selectivity and yields.

There is a need for catalysts capable of forming alkene-substituted-cyclobutanes, such as vinylcyclobutane, at commercially useful conditions. In particular, there is a need to develop new and improved iron-containing catalysts with enhanced solubility capable of forming alkene-substituted-cyclobutanes, such as vinylcyclobutane, at high selectivity and high activity.

References of interest include: Russell, S. K.; Lobkovsky, E.; Chirik, P. J., Iron-Catalyzed Intermolecular [2π+2π] Cycloaddition. J Am Chem Soc 2011, 133 (23), 8858-8861; Kennedy, C. R.; Zhong, H.; Joannou, M. V.; Chirik, P. J., Pyridine(diimine) Iron Diene Complexes Relevant to Catalytic [2+2]-Cycloaddition Reactions. Advanced Synthesis & Catalysis 2020, 362 (2), 404-416; Hoyt, J. M.; Schmidt, V. A.; Tondreau, A. M.; Chirik, P. J., Iron-catalyzed intermolecular [2+2] cycloadditions of unactivated alkenes. Science 2015, 349 (6251), 960-963; Morris, D. M.; Quintana, R. L.; Harvey, B. G., High-Performance Jet Fuels Derived from Bio-Based Alkenes by Iron-Catalyzed [2+2] Cycloaddition. Chem Sus Chem 2019, 12, 1646-1652; Kennedy, C. R.; Zhong, H.; Macaulay, R. L.; Chirik, P. J., Regio- and Diastereoselective Iron-Catalyzed [4+4]-Cycloaddition of 1,3-Dienes. J Am Chem Soc 2019, 141 (21), 8557-8573; Joannou, M. V.; Hoyt, J. M.; Chirik, P. J., Investigations into the Mechanism of Inter- and Intramolecular Iron-Catalyzed [2+2] Cycloaddition of Alkenes. J Am Chem Soc 2020, 142 (11), 5314-5330; Kennedy, C. R.; Joannou, M. V.; Steves, J. E.; Hoyt, J. M.; Kovel, C. B.; Chirik, P. J., Iron-Catalyzed Vinylsilane Dimerization and Cross-Cycloadditions with 1,3-Dienes: Probing the Origins of Chemo- and Regioselectivity. ACS Catal 2021, 11 (3), 1368-1379; Beromi, M. M.; Kennedy, C. R.; Younker, J. M.; Carpenter, A. E.; Mattler, S. J.; Throckmorton, J. A.; Chirik, P. J., Iron-catalysed synthesis and chemical recycling of telechelic 1,3-enchained oligocyclobutanes. 5 Nat Chem 2021, 13, 156-162; Beromi, M. M.; Younker, J. M.; Zhong, H.; Pabst, T. P.; Chirik, P. J., Catalyst Design Principles Enabling Intermolecular Alkene-Diene [2+2] Cycloaddition and Depolymerization Reactions. J Am Chem Soc 2021, 143 (42), 17793-17805; U.S. Pat. Nos. 8,236,915; 10,604,711; 11,001,667; WO 2021/154931; Schmidt, V. A.; Kennedy, C. R.; Bezdek, M. J.; Chirik, P. J., Selective [1,4]-Hydrovinylation of 1,3-Dienes with Unactivated Olefins Enabled by Iron Diimine Catalysts. J Am Chem Soc 2018, 140 (9), 3443-3453; Bouwkamp, M. W.; Bart, S. C.; Hawrelak, E. J.; Trovitch, R. J.; Lobkovsky, E.; Chirik, P. J., Square planar bis(imino)pyridine iron halide and alkyl complexes. Chem Commun 2005, 3406-3408; Bouwkamp, M. W.; Bowman, A. C.; Lobkovsky, E.; Chirik, P. J., Iron-Catalyzed [2π+2π] Cycloaddition of a,o-Dienes: The Importance of Redox-Active Supporting Ligands. J Am Chem Soc 2006, 128 (41), 13340-13341; Russell, S. K.; Darmon, J. M.; Lobkovsky, E.; Chirik, P. J., Synthesis of Aryl-Substituted Bis(imino)pyridine Iron Dinitrogen Complexes. Inorg Chem 2010, 49 (6), 2782-2792; and Hoyt, J. M. Iron Catalyzed C—X (X═C, Si, B, H) Bond Forming Reactions. Ph.D. Dissertation, Princeton University, 2015.

### BRIEF DESCRIPTION OF THE FIGURE

The Figure depicts an illustrative chromatogram of a sample containing vinylcyclobutane (VCB), butadiene, traces of cis-1,4-hexadiene (cis-1,4-HD), and other components, diluted with dichloromethane.

### SUMMARY

The present disclosure provides a catalyst compound represented by Formulas (I), (II), or (III):

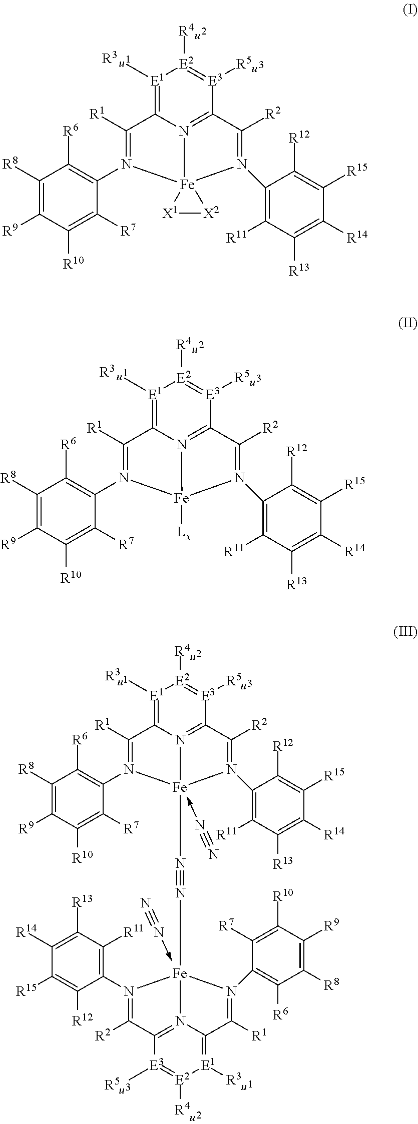

wherein:

each of R¹ and R² is independently a C₄-C₂₂ linear, branched or cyclic alkyl or alkenyl, wherein each of R¹ and R² is optionally substituted by halogen, —OR¹⁶, —NR¹⁷₂, or —SiR¹⁸₃;

each of X¹ and X² is independently a C₁ to C₂₀ hydrocarbyl where X¹ and X² form either a diene or an alkenyl diradical, optionally substituted by halogen or —SiR¹⁸₃;

each of R³, R⁴, R⁵, R⁸, R⁹, R¹⁰, R¹³, R¹⁴, and R¹⁵ is independently hydrogen, C₁-C₂₂ alkyl, C₂-C₂₂ alkenyl, C₆-C₂₂ aryl, C₇-C₃₀ arylalkyl, —OR¹⁶, —NR¹⁷₂, halogen, —SiR¹⁸₃ or five-, six-or seven-membered heterocyclic ring comprising at least one atom selected from the group consisting of N, P, O and S; wherein R³, R⁴, R⁵, R⁸, R⁹, R¹⁰, R¹³, R¹⁴, and R¹⁵ are optionally substituted by halogen, —OR¹⁶, —NR¹⁷₂, or —SiR¹⁸₃; wherein R³ optionally bonds with R⁴, R⁴ optionally bonds with R⁵, R⁷ optionally bonds with R¹⁰, R¹⁰ optionally bonds with R⁹, R⁹ optionally bonds with R⁸, R⁸ optionally bonds with R⁶, R¹² optionally bonds with R¹⁵, R¹⁵ optionally bonds with R¹⁴, R¹⁴ optionally bonds with R¹³, and R¹³ optionally bonds with R¹¹, in each case to independently form a five-, six- or seven-membered carbocyclic or heterocyclic ring, the heterocyclic ring comprising at least one atom from the group consisting of N, P, O and S;

each of R⁶, R⁷, R¹¹, and R¹² is independently hydrogen, C₁-C₂₂ alkyl, C₂-C₂₂ alkenyl, C₆-C₂₂ aryl, C₇-C₃₀ arylalkyl, a heteroatom or a heteroatom-containing group (such as —OR¹⁶, —NR¹⁷₂, halogen, —SiR¹⁸₃ or five-, six- or seven-membered heterocyclic ring comprising at least one atom selected from the group consisting of N, P, O and S); wherein R⁶, R⁷, R¹¹, and R¹² are optionally substituted by halogen, —OR¹⁶, —NR¹⁷₂, or —SiR¹⁸₃, wherein R⁶ optionally bonds with R⁸, R⁷ optionally bonds with R¹⁰, R¹¹ optionally bonds with R¹³, or R¹⁵ optionally bonds with R¹² in each case to independently form a five-, six- or seven-membered carbocyclic or heterocyclic ring, the heterocyclic ring comprising at least one atom from the group consisting of N, P, O and S;

each of R¹⁶, R¹⁷, and R¹⁸ is independently hydrogen, C₁-C₂₂ alkyl, C₂-C₂₂ alkenyl, C₆-C₂₂ aryl, C₇-C₃₀ arylalkyl, or —SiR¹⁹₃, wherein each R¹⁶, R¹⁷, and R¹⁸ is independently optionally substituted by halogen, or two R¹⁷ radicals optionally bond to form a five- or six-membered ring, or two R¹⁸ radicals optionally bond to form a five- or six-membered ring;

each R¹⁹ is independently hydrogen, C₁-C₂₂ alkyl, C₂-C₂₂ alkenyl, C₆-C₂₂ aryl, C₇-C₃₀ arylalkyl, or two R¹⁹ radicals optionally bond to form a five- or six-membered ring; [0013] each of E¹, E², and E³ is independently carbon, nitrogen or phosphorus;

each of u¹, u², and u³ is 0 when E¹, E², or E³ is nitrogen or phosphorus, and each of u¹, u², and u³ is 1 when E¹, E², or E³ is carbon;

L is either a dinitrogen group when x is 2 and a single L is optionally bonded to a second metal center, or is a methyl group when x is 1.

The present disclosure further provides a process to produce substituted-cyclobutanes, such as vinylcyclobutane, by contacting one or more olefins with a catalyst compound represented by Formulas (I), (II), or (III) above.

### DETAILED DESCRIPTION

Catalyst compounds of the present disclosure are iron-containing compounds including an 2,6-diimino(heteroaryl) ligand and diene adduct. Catalyst compounds of the present disclosure can be soluble in hydrophobic (nonpolar) solvents and are capable of forming substituted-cyclobutanes, such as vinylcyclobutane. By “soluble”, it is meant the catalyst exhibits a solubility in aliphatic hydrocarbons of equal to or greater than 0.5 wt %. It has been surprisingly and unexpectedly discovered that the introduction of a functional group, particularly one that is solubilizing, to the catalysts Formulas (I) and (II) allows for the convenient isolation of catalyst without the need for purification by recrystallization. It has also been surprisingly and unexpectedly discovered that the introduction of one or more functional groups (R¹ and/or R²), particularly one that is solubilizing, to the catalysts at the imine carbon results in increased thermal stability and higher catalyst activity for cycloaddition at elevated temperatures. By “elevated temperatures”, it is meant a commercial bulk temperature above ambient temperature.

The term “complex” is used to describe molecules in which an ancillary ligand is coordinated to a central transition metal atom. The ligand is bulky and stably bonded to the transition metal so as to maintain its influence during use of the catalyst, such as cycloaddition. The ligand may be coordinated to the transition metal by covalent bond(s) and or electron donation coordination or intermediate bonds.

For the purposes of the present disclosure, the numbering scheme for the Periodic Table Groups is used as described in Chemical and Engineering News, v.63(5), pg. 27 (1985). Therefore, a “Group 8 metal” is an element from Group 8 of the Periodic Table, e.g., Fe.

The following abbreviations may be used herein: Me is methyl, Et is ethyl, Ph is phenyl, tBu is tertiary butyl, nBu is normal butyl, iPr is isopropyl, and THF (also referred to as thf) is tetrahydrofuran, NMR is nuclear magnetic resonance, t is time, s is second, h is hour, psi is pounds per square inch, psig is pounds per square inch gauge, psid is pounds per square inch differential, equiv is equivalent, rpm is rotation per minute.

The terms “substituent,” “radical,” “group,” and “moiety” may be used interchangeably. Unless otherwise specified, the term “radical” refers to an X-type ligand in the context of the covalent model for electron counting and does not refer to the distribution of an electron or electrons within a complex.

An “olefin,” alternatively referred to as “alkene,” is a linear, branched, or cyclic compound of carbon and hydrogen having at least one double bond. The term “olefin” broadly refers to any and all diolefins, alpha-olefins and conjugated dienes.

A “diolefin,” alternatively referred to as “diene,” is a linear, branched, or cyclic compound of carbon and hydrogen having at least two double bonds.

The term “alpha-olefin” refers to an olefin having a terminal carbon-to-carbon double bond in the structure thereof ((R′R″′)—C═CH₂, where R″ and R″′ can be independently hydrogen or any hydrocarbyl group; such as R″ is hydrogen and R′″ is an alkyl group). A “linear alpha-olefin” is an alpha-olefin defined in this paragraph wherein R″ is hydrogen, and R is hydrogen or a linear alkyl group. For the purposes of the present disclosure, ethylene shall be considered an alpha-olefin.

The term “conjugated diene” refers to a diolefin having at least two alternating carbon-to-carbon double bonds.

As used herein, and unless otherwise specified, the term “C.” means hydrocarbon(s) having n carbon atom(s) per molecule, wherein n is a positive integer. The term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated and or unsaturated), including mixtures of hydrocarbon compounds having different values of n. Likewise, a “Cₘ-C_{y}” group or compound refers to a group or compound including carbon atoms at a total number thereof from m to y. Thus, a C₁-C₅₀ alkyl group refers to an alkyl group including carbon atoms at a total number thereof from about 1 to about 50.

Unless otherwise indicated, (e.g., the definition of “substituted hydrocarbyl”, “substituted aromatic”, etc.), the term “substituted” means that at least one hydrogen atom has been replaced with at least one non-hydrogen group, such as a hydrocarbyl group, a heteroatom, or a heteroatom containing group, such as halogen (such as Br, Cl, F or I) or at least one functional group such as —NR*₂, —OR*, —SeR*, —TeR*, —PR*₂, —AsR*₂, —SbR*₂, —SR*, —BR*₂, —SiR*₃, —GeR*₃, —SnR*₃, —PbR*₃, where each R* is independently a hydrocarbyl or halocarbyl radical, and two or more R* may join together to form a substituted or unsubstituted completely saturated, partially unsaturated, or aromatic cyclic or polycyclic ring structure), or where at least one heteroatom has been inserted within a hydrocarbyl ring.

The term “substituted hydrocarbyl” means a hydrocarbyl radical in which at least one hydrogen atom of the hydrocarbyl radical has been substituted with at least one heteroatom (such as halogen, e.g., Br, Cl, F or I) or heteroatom-containing group (such as a functional group, e.g., —NR*₂, —OR*, —SeR*, —TeR*, —PR*₂, —AsR*₂, —SbR*₂, —SR*, —BR*₂, —SiR*₃, —GeR*₃, —SnR*₃, —PbR*₃, where each R* is independently a hydrocarbyl or halocarbyl radical, and two or more R* may join together to form a substituted or unsubstituted completely saturated, partially unsaturated, or aromatic cyclic or polycyclic ring structure), or where at least one heteroatom has been inserted within a hydrocarbyl ring. The term “hydrocarbyl substituted phenyl” means a phenyl group having 1, 2, 3, 4 or 5 hydrogen groups replaced by a hydrocarbyl or substituted hydrocarbyl group. For example, the “hydrocarbyl substituted phenyl” group can be represented by the formula:

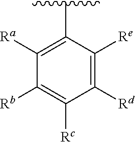

where each of R^{a}, R^{b}, R^{c}, R^{d}, and R^{e} can be independently selected from hydrogen, C₁-C₄₀ hydrocarbyl or C₁-C₄₀ substituted hydrocarbyl, a heteroatom or a heteroatom-containing group (provided that at least one of R^{a}, R^{b}, R^{c}, R^{d}, and R^{e} is not H), or two or more of R^{a}, R^{b}, R^{c}, R^{d}, and R^{e} can be joined together to form a C₄-C₆₂ cyclic or polycyclic hydrocarbyl ring structure, or a combination thereof.

The term “substituted aromatic,” means an aromatic group having 1 or more hydrogen groups replaced by a hydrocarbyl, substituted hydrocarbyl, heteroatom or heteroatom containing group.

The term “substituted phenyl,” mean a phenyl group having 1 or more hydrogen groups replaced by a hydrocarbyl, substituted hydrocarbyl, heteroatom or heteroatom containing group.

The term “substituted naphthyl,” means a naphthyl group having 1 or more hydrogen groups replaced by a hydrocarbyl, substituted hydrocarbyl, heteroatom or heteroatom containing group.

The term “substituted benzyl” means a benzyl group having 1 or more hydrogen groups replaced by a hydrocarbyl, substituted hydrocarbyl, heteroatom or heteroatom containing group, such as a substituted benzyl group is represented by the formula:

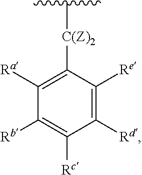

where each of R^{a}′, R^{b}′, R^{c}′, R^{d}′, and R^{e}′ and Z is independently selected from hydrogen, C₁-C₄₀ hydrocarbyl or C₁-C₄₀ substituted hydrocarbyl, a heteroatom or a heteroatom-containing group (provided that at least one of R^{a}′, R^{b}′, R^{c}′, R^{d}′, and R^{e}′ and Z is not H), or two or more of R^{a}′, R^{b}′, R^{c}′, R^{d}′, and R^{e}′ and Z are joined together to form a C₄-C₆₂ cyclic or polycyclic ring structure, or a combination thereof.

The terms “alkoxy” and “alkoxide” mean an alkyl or aryl group bound to an oxygen atom, such as an alkyl ether or aryl ether group/radical connected to an oxygen atom and can include those where the alkyl/aryl group is a C₁ to C₁₀ hydrocarbyl. The alkyl group may be straight chain, branched, or cyclic. The alkyl group may be saturated or unsaturated. Examples of suitable alkoxy radicals can include methoxy, ethoxy, n-propoxy, iso-propoxy, n-butoxy, iso-butoxy, sec-butoxy, tert-butoxy, phenoxyl.

The term “alkenyl” means a straight-chain, branched-chain, or cyclic hydrocarbon radical having one or more double bonds. These alkenyl radicals may be optionally substituted. Examples of suitable alkenyl radicals can include ethenyl, propenyl, allyl, 1,4-butadienyl, cyclopropenyl, cyclobutenyl, cyclopentenyl, cyclohexenyl, cycloctenyl, including their substituted analogues.

The term “alkenyl diradical” means an alkenyl radical having two unpaired electrons. These alkenyl diradicals may be optionally substituted. An example of a suitable alkenyl diradical is 2-hexene-1,6-diyl and its substituted analogues, which can be represented by any of the following formulas:

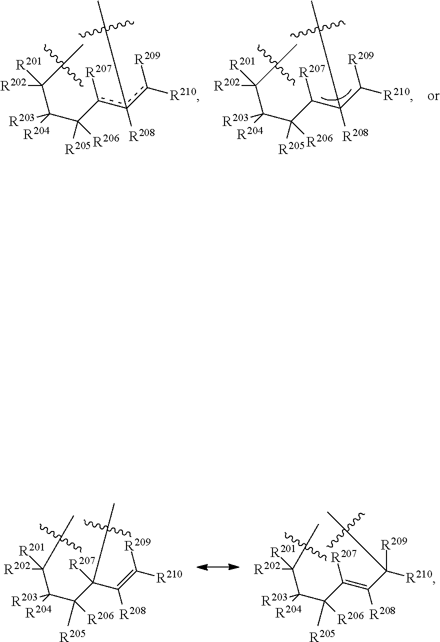

where each of R²⁰¹, R²⁰², R²⁰³, R²⁰⁴, R²⁰⁵, R²⁰⁶, R²⁰⁷, R²⁰⁸, R²⁰⁹, and R²¹⁰ can be independently selected from hydrogen, C₁-C₄₀ hydrocarbyl or C₁-C₄₀ substituted hydrocarbyl, a heteroatom or a heteroatom-containing group, or two or more of R²⁰¹, R²⁰², R²⁰³, R²⁰⁴, R²⁰⁵, R²⁰⁶, R²⁰⁷, R²⁰⁸, R²⁰⁹, and R²¹⁰ can be joined together to form a C₄-C₆₂cyclic or polycyclic hydrocarbyl ring structure, or a combination thereof.

The terms “alkyl radical,” and “alkyl” are used interchangeably throughout this disclosure. For purposes of this disclosure, “alkyl radical” is defined to be C₁-C₁₀₀ alkyls that may be linear, branched, or cyclic. Examples of such radicals can include methyl, ethyl, n-propyl, isopropyl, n-butyl, isobutyl, sec-butyl, tert-butyl, pentyl, iso-amyl, hexyl, octyl, cyclopropyl, cyclobutyl, cyclopentyl, cyclohexyl, cyclooctyl, including their substituted analogues. Substituted alkyl radicals are radicals in which at least one hydrogen atom of the alkyl radical has been substituted with at least a non-hydrogen group, such as a hydrocarbyl group, a heteroatom, or a heteroatom containing group, such as halogen (such as Br, Cl, F or I) or at least one functional group such as —NR*₂, —OR*, —SeR*, —TeR*, —PR*₂, —AsR*₂, —SbR*₂, —SR*, —BR*₂, —SiR*₃, —GeR*₃, —SnR*₃, —PbR*₃, or —(CH₂)— SiR*₃, and each R* is independently a hydrocarbyl or halocarbyl radical, and two or more R* may join together to form a substituted or unsubstituted completely saturated, partially unsaturated, or aromatic cyclic or polycyclic ring structure), or where at least one heteroatom has been inserted within a hydrocarbyl ring.

The term “aryl” or “aryl group” means an aromatic ring and the substituted variants thereof, such as phenyl, 2-methyl-phenyl, xylyl, 4-bromo-xylyl. Likewise, heteroaryl means an aryl group where a ring carbon atom (or two or three ring carbon atoms) has been replaced with a heteroatom, such as N, O, or S. As used herein, the term “aromatic” also refers to pseudoaromatic heterocycles which are heterocyclic substituents that have similar properties and structures (nearly planar) to aromatic heterocyclic ligands, but are not by definition aromatic; likewise the term aromatic also refers to substituted aromatics.

The term “arylalkyl” means an aryl group where a hydrogen has been replaced with an alkyl or substituted alkyl group. For example, 3,5′-di-tert-butyl-phenyl indenyl is an indene substituted with an arylalkyl group. When an arylalkyl group is a substituent on another group, it is bound to that group via the aryl.

Where isomers of a named alkyl, alkenyl, alkoxy, or aryl group exist (e.g., n-butyl, iso-butyl, sec-butyl, and tert-butyl) reference to one member of the group (e.g., n-butyl) shall expressly disclose the remaining isomers (e.g., iso-butyl, sec-butyl, and tert-butyl) in the family. Likewise, reference to an alkyl, alkenyl, alkoxy, or aryl group without specifying a particular isomer (e.g., butyl) expressly discloses all isomers (e.g., n-butyl, iso-butyl, sec-butyl, and tert-butyl).

The term “ring atom” means an atom that is part of a cyclic ring structure. By this definition, a benzyl group has six ring atoms and tetrahydrofuran has 5 ring atoms.

A heterocyclic ring is a ring having a heteroatom in the ring structure as opposed to a heteroatom substituted ring where a hydrogen on a ring atom is replaced with a heteroatom. For example, tetrahydrofuran is a heterocyclic ring and 4-N,N-dimethylamino-phenyl is a heteroatom-substituted ring. Other examples of heterocycles may include pyridine, imidazole, and thiazole.

The terms “hydrocarbyl radical,” “hydrocarbyl group,” or “hydrocarbyl” may be used interchangeably and are defined to mean a group including hydrogen and carbon atoms only. For example, a hydrocarbyl can be a C₁-C₁₀₀ radical that may be linear, branched, or cyclic, and when cyclic, aromatic or non-aromatic. Examples of such radicals may include, but are not limited to, alkyl groups such as methyl, ethyl, n-propyl, isopropyl, n-butyl, isobutyl, sec-butyl, tert-butyl, pentyl, iso-amyl, hexyl, octyl cyclopropyl, cyclobutyl, cyclopentyl, cyclohexyl, cyclooctyl, and aryl groups, such as phenyl, benzyl, naphthyl.

The terms “catalyst compound”, “catalyst complex”, “transition metal complex”, and “transition metal compound” are used interchangeably.

A “Lewis base” is a neutral ligand that donates one or more pairs of electrons to a metal ion. Examples of Lewis bases include ethylether, trimethylamine, pyridine, tetrahydrofuran, dimethylsulfide, and triphenylphosphine. The term “heterocyclic Lewis base” refers to Lewis bases that are also heterocycles. Examples of heterocyclic Lewis bases include pyridine, imidazole, thiazole, and furan.

The term “continuous” means a system that operates without interruption or cessation. For example, a continuous process to produce a product would be one where the reactants are continually introduced into one or more reactors and product is continually withdrawn.

Transition Metal Complexes

In at least one embodiment, the iron-containing catalyst can have heteroaryl and diene ligands. In particular, the present disclosure relates to iron-containing catalyst compounds having a 2,6-diimino(heteroaryl) ligand and either a diene adduct or dinitrogen group. The iron-containing catalyst can be represented by Formulas (I), (II), or (III) below:

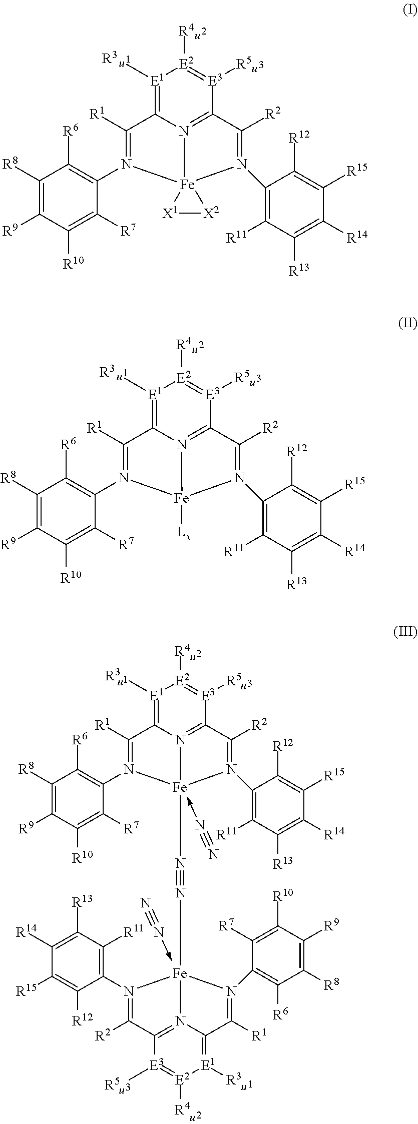

wherein:

each of R¹ and R² is independently a C₄-C₂₂ linear, branched or cyclic alkyl or alkenyl, wherein each of R¹ and R² is optionally substituted by halogen, —OR¹⁶, —NR¹⁷₂, or —SiR¹⁸₃;

each of X¹ and X² is independently a C₁ to C₂₀ hydrocarbyl where X¹ and X² form either a diene (such as an s-trans conjugated) diene or an alkenyl diradical, optionally substituted by halogen or —SiR¹⁸₃;

each of R³, R⁴, R⁵, R⁸, R⁹, R¹⁰, R¹³, R¹⁴, and R¹⁵ is independently hydrogen, C₁-C₂₂ alkyl, C₂-C₂₂ alkenyl, C₆-C₂₂ aryl, C₇-C₃₀ arylalkyl, —OR¹⁶, —NR¹⁷₂, halogen, —SiR¹⁸₃ or five-, six-or seven-membered heterocyclic ring comprising at least one atom selected from the group consisting of N, P, O and S; wherein R³, R⁴, R⁵, R⁸, R⁹, R¹⁰, R¹³, R¹⁴, and R¹⁵ are optionally substituted by halogen, —OR¹⁶, —NR¹⁷₂, or —SiR¹⁸₃; wherein R³ optionally bonds with R⁴, R⁴ optionally bonds with R⁵, R⁷ optionally bonds with R¹⁰, R¹⁰ optionally bonds with R⁹, R⁹ optionally bonds with R⁸, R⁸ optionally bonds with R⁶, R¹² optionally bonds with R¹⁵, R¹⁵ optionally bonds with R¹⁴, R¹⁴ optionally bonds with R¹³, and R¹³ optionally bonds with R″, in each case to independently form a five-, six- or seven-membered carbocyclic or heterocyclic ring, the heterocyclic ring comprising at least one atom from the group consisting of N, P, O and S;

each of R⁶, R⁷, R″, and R¹² is independently hydrogen, C₁-C₂₂ alkyl, C₂-C₂₂ alkenyl, C₆-C₂₂ aryl, C₇-C₃₀ arylalkyl, a heteroatom or a heteroatom-containing group (such as —OR¹⁶, —NR¹⁷₂, halogen, —SiR¹⁸₃ or five-, six- or seven-membered heterocyclic ring including at least one atom selected from the group consisting of N, P, O and S); wherein R⁶, R⁷, R¹¹, and R¹² are optionally substituted by halogen, —OR¹⁶, —NR¹⁷₂, or —SiR¹⁸₃, wherein R⁶ optionally bonds with R⁸, R⁷ optionally bonds with R¹⁰, R¹¹ optionally bonds with R¹³, or R¹⁵ optionally bonds with R¹² in each case to independently form a five-, six- or seven-membered carbocyclic or heterocyclic ring, the heterocyclic ring including at least one atom from the group consisting of N, P, O and S;

each of R¹⁶, R¹⁷, and R¹⁸ is independently hydrogen, C₁-C₂₂ alkyl, C₂-C₂₂ alkenyl, C₆-C₂₂ aryl, C₇-C₃₀ arylalkyl, or —SiR¹⁹₃, wherein each R¹⁶, R¹⁷, and R¹⁸ is independently optionally substituted by halogen, or two R¹⁷ radicals optionally bond to form a five- or six-membered ring, or two R¹⁸ radicals optionally bond to form a five- or six-membered ring;

each R¹⁹ is independently hydrogen, C₁-C₂₂ alkyl, C₂-C₂₂ alkenyl, C₆-C₂₂ aryl, C₇-C₃₀ arylalkyl, or two R¹⁹ radicals optionally bond to form a five- or six-membered ring;

each of E¹, E², and E³ is independently carbon, nitrogen or phosphorus;

each of u¹, u², and u³ is 0 when E¹, E², or E³ is nitrogen or phosphorus, and each of u¹, u², and u³ is 1 when E¹, E², or E³ is carbon;

L is either a dinitrogen group when x is 2 and a single L is optionally bonded to a second metal center, or L is a methyl group when x is 1.

In at least one embodiment, each of R¹ and R² is independently C₄-C₂₂ alkyl. One or more of R¹ and R² may be independently selected from butyl, pentyl, hexyl, heptyl, octyl, nonyl, decyl, undecyl, dodecyl, or a linear, branched or cyclic isomer thereof). In at least one embodiment, R¹ and R² are n-butyl, n-pentyl, n-hexyl, n-heptyl, n-octyl, n-nonyl, n-decyl, n-undecyl, n-dodecyl, n-hexadecyl, n-octadecyl, cyclobutyl, cyclopentyl, cyclohexyl, cycloheptyl, cyclooctyl, cyclononyl, cyclodecyl, cycloundecyl, cyclododecyl, cyclohexadecyl, cyclooctadecyl, iso-butyl, iso-pentyl, iso-hexyl, iso-heptyl, iso-octyl, iso-nonyl, iso-decyl, iso-undecyl, iso-dodecyl, iso-hexadecyl, and iso-octadecyl.

In at least one embodiment, each of R⁶, R⁷, R¹¹ and R¹² are independently selected from methyl, ethyl, propyl, butyl, pentyl, hexyl, heptyl, octyl, nonyl, decyl, dimethyl-pentyl, tert-butyl, isopropyl, cyclopentyl or isomers thereof.

Each of R¹⁶ and R¹⁷ is independently hydrogen, C₁-C₂₂-alkyl, C₂-C₂₂-alkenyl, C₆-C₂₂-aryl, C₇-C₃₀ arylalkyl, or —SiR¹⁸₃, wherein R¹⁶ and or R¹⁷ is optionally substituted by halogen, or R¹⁷ radicals optionally bond to form a five- or six-membered ring. Each R¹⁸ is independently hydrogen, C₁-C₂₂-alkyl, C₂-C₂₂-alkenyl, C₆-C₂₂-aryl, C₇-C₃₀ arylalkyl, or two R¹⁸ radicals optionally bond to form a five- or six-membered ring.

In at least one embodiment, each of R³, R⁴, R⁵ is independently selected from hydrogen, methyl, ethyl, propyl, butyl, pentyl, hexyl, heptyl, octyl, nonyl, decyl, dimethyl-pentyl, tert-butyl, isopropyl, or isomers thereof, such as R³, R⁴, and R⁵ are hydrogen.

Each of R⁶, R⁷, R⁸, R⁹, R¹⁰, R¹¹, R¹², R¹³, R¹⁴, and R¹⁵ can be independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as perfluoropropyl, perfluorobutyl, perfluoroethyl, perfluoromethyl), substituted hydrocarbyl radicals and all isomers of substituted hydrocarbyl radicals including trimethylsilylpropyl, trimethylsilylmethyl, trimethylsilylethyl, phenyl, or all isomers of hydrocarbyl substituted phenyl including methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, or dipropylmethylphenyl, or isomers thereof.

Each of R³, R⁴, and R⁵ can be independently hydrogen or C₁-C₂₂-alkyl.

In at least one embodiment, E¹, E², and E³ are carbon, and each of R³, R⁴, and R⁵ is independently hydrogen or C₁-C₂₂-alkyl.

In at least one embodiment, E¹, E², and E³ are carbon, and each of R³, R⁴, and R⁵ is hydrogen.

In another embodiment, each of R¹ and R² are independently n-butyl, n-pentyl or n-hexyl, E¹, E², and E³ are carbon, and R³, R⁴, and R⁵ are hydrogen.

In another embodiment, both R¹ and R² are n-butyl, n-pentyl or n-hexyl, E¹, E², and E³ are carbon, and R³, R⁴, and R⁵ are hydrogen.

In another embodiment, R¹ and R² are n-butyl, E¹, E², and E³ are carbon, and R³, R⁴, and R⁵ are hydrogen.

In at least one embodiment R⁶, R⁷, R¹¹, and R¹² are independently selected from methyl, ethyl, propyl, butyl, —CF₃, or Cl.

In at least one embodiment, R¹ and R² are n-butyl, E¹, E², and E³ are carbon, and R³, R⁴, and R⁵ are hydrogen, and R⁶, R⁷, R¹¹, and R¹² are methyl.

In at least one embodiment, each of X¹ and X² is independently a C₁ to C₂₀ hydrocarbyl where X¹ and X² form a conjugated diene, such as an s-trans conjugated diene, such as a C₄ to C₄₀ diene, such as a C₄ to C₁2 diene. For example, X¹ and X² form 1,3-butadiene, piperylene, isoprene, or myrcene.

In at least one embodiment, the catalyst compound represented by Formula (I) is one or more of:

[2,6-Bis(1-(2,6-dimethylphenylimino)pentyl)pyridine] iron butadiene;
[2,6-Bis(1-(2,6-dimethylphenylimino)pentyl)pyridine] iron isoprene;
[2,6-Bis(1-(2,6-dimethylphenylimino)pentyl)pyridine] iron myrcene;
[2,6-Bis(1-(2,6-dimethylphenylimino)pentyl)pyridine] iron 2-hexene-1,6-diyl;
[2,6-Bis(1-(2,6-dimethylphenylimino)hexyl)pyridine] iron butadiene;
[2,6-Bis(1-(2,6-dimethylphenylimino)hexyl)pyridine] iron isoprene;
[2,6-Bis(1-(2,6-dimethylphenylimino)hexyl)pyridine] iron myrcene;
[2,6-Bis(1-(2,6-dimethylphenylimino)hexyl)pyridine] iron 2-hexene-1,6-diyl;
[2,6-Bis(1-(2,6-dimethylphenylimino)heptyl)pyridine] iron butadiene;
[2,6-Bis(1-(2,6-dimethylphenylimino)heptyl)pyridine] iron isoprene;
[2,6-Bis(1-(2,6-dimethylphenylimino)heptyl)pyridine] iron myrcene;
[2,6-Bis(1-(2,6-dimethylphenylimino)heptyl)pyridine] iron 2-hexene-1,6-diyl;
[2,6-Bis(1-(2,6-dimethylphenylimino)octyl)pyridine] iron butadiene;
[2,6-Bis(1-(2,6-dimethylphenylimino)octyl)pyridine] iron isoprene;
[2,6-Bis(1-(2,6-dimethylphenylimino)octyl)pyridine] iron myrcene;
[2,6-Bis(1-(2,6-dimethylphenylimino)octyl)pyridine] iron 2-hexene-1,6-diyl;
[2,6-Bis(1-(2,6-dimethylphenylimino)nonyl)pyridine] iron butadiene;
[2,6-Bis(1-(2,6-dimethylphenylimino)nonyl)pyridine] iron isoprene;
[2,6-Bis(1-(2,6-dimethylphenylimino)nonyl)pyridine] iron myrcene; and
[2,6-Bis(1-(2,6-dimethylphenylimino)nonyl)pyridine] iron 2-hexene-1,6-diyl.

With reference to catalyst Formula (I), specific catalyst structures can include the following structures I-A through I-H:

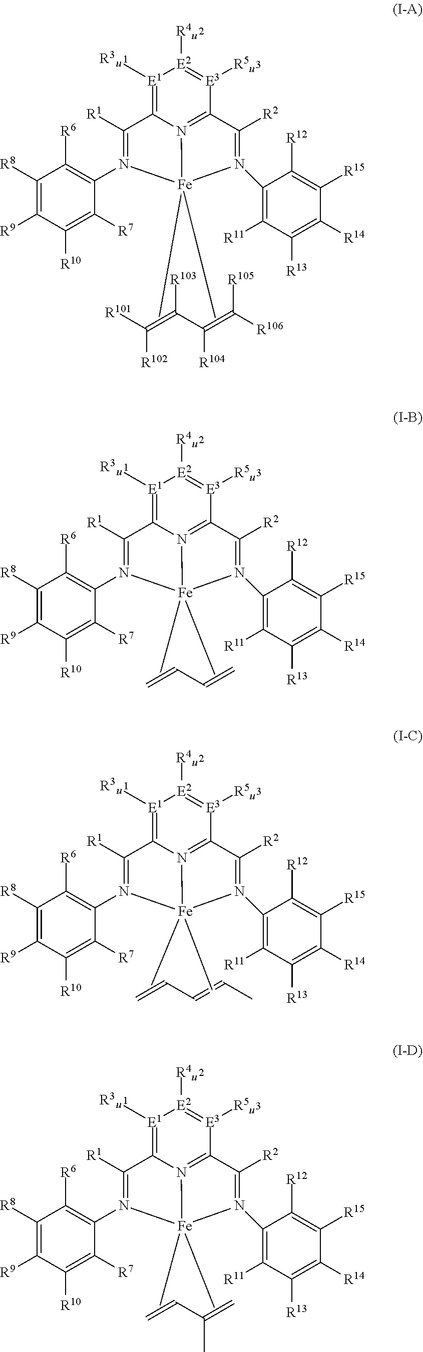

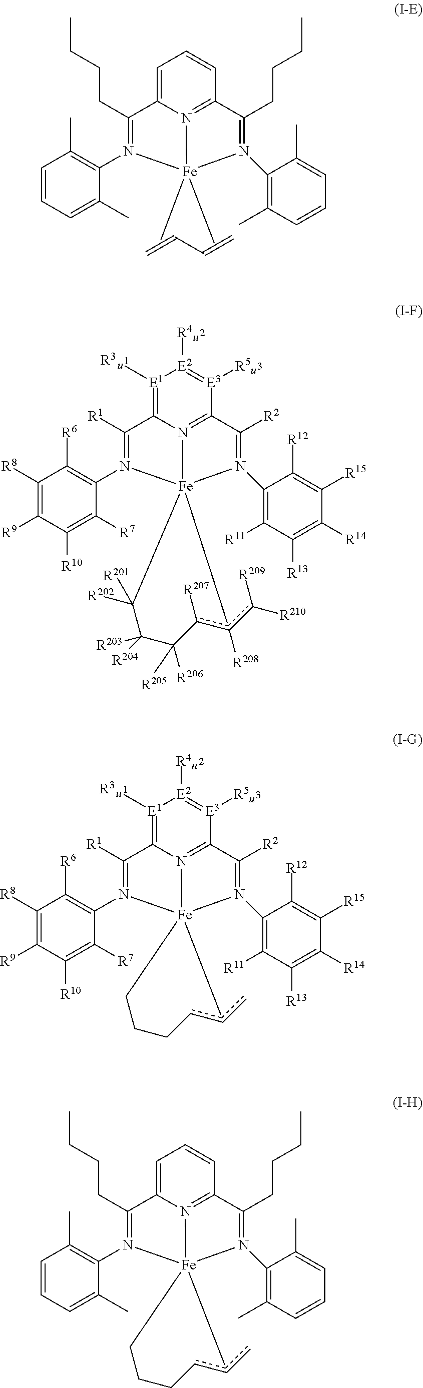

With reference to catalyst Formula (II), specific catalyst structures can include the following structures II-A through II-B:

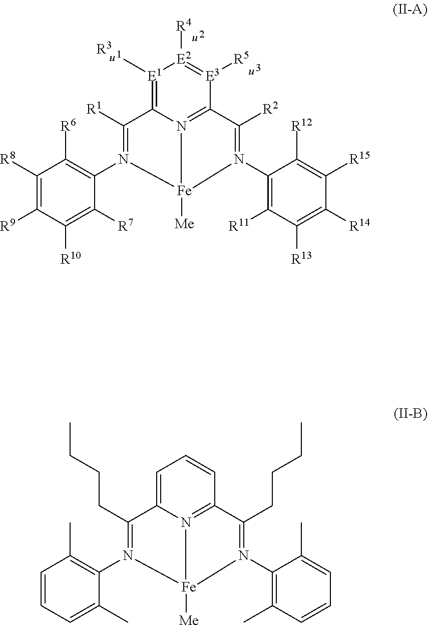

### Methods to Prepare the Catalyst Compounds.

The following is a generic description to prepare a catalyst compound described herein and further exemplified in the examples. Scheme 1 illustrates various synthetic routes to prepare iron cycloaddition catalysts. All air sensitive syntheses can be carried out in nitrogen purged dry boxes. All solvents are available from commercial sources. Substituted anilines; substituted pyridines; Grignard reagents; iron (II) chloride; and p-toluenesulfonic acid may be available from commercial sources. A mixture of a 2,6-dicarbonyl(heteroarene) (e.g., 2,6-divaleroylpyridine), an aniline (e.g., 2,6-dimethylaniline), and a catalytic amount of acid (e.g., p-toluenesulfonic acid) in toluene can be refluxed using a Dean-Stark apparatus to form a 2,6-diimino(heteroaryl) ligand. The resulting compound can then be treated with iron(II) halide (e.g., iron(II) chloride) to form an iron dihalide complex. The resulting iron dihalide complex can then be converted into several forms, each of which can serve as a catalyst for [2π+2π] cycloaddition processes.

The iron dihalide complex can be treated with a reductant (e.g. sodium mercury amalgam, sodium naphthalenide, sodium metal, potassium graphite, etc.) in the presence of dinitrogen to form an iron dinitrogen complex, represented by Formula (II) where L is dinitrogen, in some cases also represented by Formula (III) when substitutions on the aniline are sufficiently less bulky to allow for dimerization. Treatment of the iron dinitrogen complex with vinylcyclobutane can provide access to an iron metallocycle complex, represented by structure I-F of Formula (I). Alternatively, treatment of the iron dinitrogen complex with a diene (e.g. butadiene) can provide access to an iron diene complex, represented by Formula (I), more specifically represented by structure I-A of Formula (I) when a conjugated diene is used.

Alternatively, the iron dihalide complex can be treated with a magnesium diene reagent (e.g. Mg(butadiene)(THF)₂) to provide access to an iron diene complex, represented by Formula (I), more specifically represented by structure I-A of Formula (I) when a conjugated diene is used. Treatment of the iron butadiene complex with vinylcyclobutane can provide access to the iron metallocycle complex.

Alternatively, the iron dihalide complex can be treated with two equivalents of methyllithium to afford the iron monomethyl complex, represented by structure II-A of Formula (II). Treatment of the iron monomethyl complex with vinylcyclobutane can provide access to an iron metallocycle complex, represented by structure I-F of Formula (I).

Alternatively, treatment of the iron monomethyl complex with a diene (e.g. butadiene) can provide access to an iron diene complex, represented by Formula (I), more specifically represented by structure I-A of Formula (I) when a conjugated diene is used. Preferably, the resulting iron complex in hydrocarbon solvent is purified by filtration, such as through Celite, to remove any insoluble impurities.

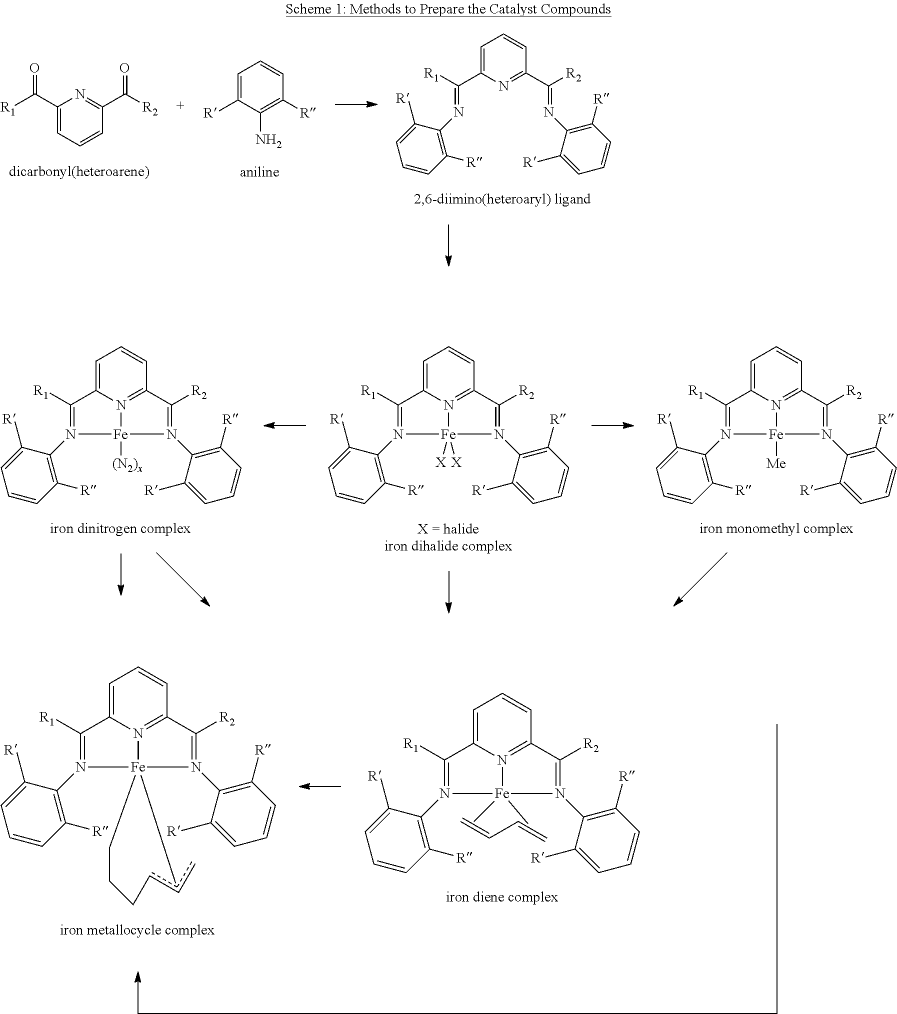

### Substituted Cyclobutane Products

The catalysts provided herein can be used to make substituted-cyclobutanes, such as vinylcyclobutanes from at least one alpha-olefin and/or at least one diene. Suitable alpha-olefins include substituted or unsubstituted C₂ to C₄₀ alpha olefins, such as C₂ to C₂₀ alpha olefins, such as C₂ to C₁₂ alpha olefins, such as ethylene, propylene, butene, pentene, hexene, heptene, octene, nonene, decene, undecene, dodecene and isomers thereof. Suitable dienes include C₄ to C₄₀ diolefins, which may be linear, branched or cyclic, and may be conjugated dienes, such as 1,3-butadiene, 1,3-hexadiene, 1,3-heptadiene, 1,3-octadiene, 1,3-nonadiene, 1,3-decadiene, 1,3-dodecadiene. Useful dienes include: butadiene, hexadiene, heptadiene, octadiene, nonadiene, decadiene, dodecadiene, substituted derivatives thereof, and isomers thereof.

The substituted-cyclobutanes, such as vinylcyclobutane, can be a typical [2π+2π]cycloaddition product, which can be represented by the following formula (F-1):

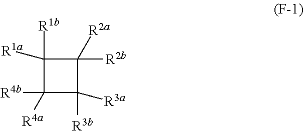

where R^{1a}, R^{1b}, R^{2a}, R^{2b}, R^{3a}, R^{3b}, R^{4a}, and R^{4b}, are each, independently, hydrogen or a substituted or unsubstituted hydrocarbyl (preferably an alkyl or alkenyl) group.

In an embodiment, when R^{3a}, R^{3b}, R^{4a}, and R^{4b} are all hydrogen, only one of Ria and R^{1b} is hydrogen, and only one of R^{2a} and R^{2b} is hydrogen, (F-1) represents a 1,2-disubstituted cyclobutane.

In an embodiment, when R^{2a}, R^{2b}, R^{4a}, and R^{4b} are all hydrogen, only one of Ria and R^{1b} is hydrogen, and only one of R^{3a} and R^{3b} is hydrogen, (F-1) represents a 1,3-disubstituted cyclobutane.

In an embodiment, when only one of Ria and R^{1b} contains a C═C directly bound to the four-membered ring, (F-1) represents an alkenyl-substituted cyclobutane.

1,2-disubstituted cyclobutanes are typically produced from the [2π+2π] cycloaddition reaction of two alpha olefins.

1,3-disubstituted cyclobutanes are typically produced from the [2π+2π] cycloaddition reaction of an alpha olefin and a conjugated diene, a diolefin (preferably containing at least one terminal olefin) and a conjugated diene, or a conjugated diene with another conjugated diene. An example of a 1,3-disubstituted cyclobutane can be represented by the following formula:

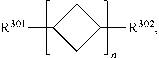

where R³⁰¹ can be independently selected from C₁-C₄₀ hydrocarbyl (such as vinyl) or C₁-C₄₀ substituted hydrocarbyl, or a heteroatom or a heteroatom-containing group; R³⁰² can be independently selected from hydrogen (provided that n is greater than 1), C₁-C₄₀ hydrocarbyl (such as vinyl) or C₁-C₄₀ substituted hydrocarbyl, or a heteroatom or a heteroatom-containing group; and n is equal to or greater than 1. 1,3-Disubstituted cyclobutanes, where R³⁰¹ and R³⁰² are both vinyl and n is variable, can be produced from the [2π+2π] cycloaddition reaction of butadiene and butadiene.

Alkenyl-substituted cyclobutanes are typically produced from the [2π+2π] cycloaddition reaction including a conjugated diene. Vinylcyclobutane is typically produced from the [2π+2π] cycloaddition reaction of ethylene and butadiene.

The substituted-cyclobutanes, such as vinylcyclobutane, can be made using a solution cycloaddition process. A solution cycloaddition is a cycloaddition process in which the cycloaddition product is dissolved in a liquid cycloaddition medium, such as an inert solvent or monomer(s) or their blends. A solution cycloaddition is typically homogeneous. A homogeneous cycloaddition is one where the cycloaddition product is dissolved in the cycloaddition medium. Such systems are not turbid. Solution cycloaddition may involve cycloaddition in a continuous reactor in which the polymer formed, the starting monomer and catalyst materials supplied are agitated to reduce or avoid concentration gradients and in which the monomer acts as a diluent or solvent or in which a hydrocarbon is used as a diluent or solvent. Suitable processes can operate at temperatures from about 0° C. to about 250° C., such as from about 20° C. to about 200° C., such as from about 35° C. to about 80° C., such as from about 50° C. to about 70° C., and or at pressures of about 0.1 MPa or more, such as 0.5 MPa or more. The upper pressure limit is not critically constrained but can be about 200 MPa or less, such as 120 MPa or less, such as 30 MPa or less. Temperature control in the reactor can be obtained by balancing the heat of cycloaddition and with reactor cooling by reactor jackets or cooling coils to cool the contents of the reactor, auto refrigeration, pre-chilled feeds, vaporization of liquid medium (diluent, reactants or solvent) or combinations of all three. Adiabatic reactors with pre-chilled feeds can also be used. The purity, type, and amount of solvent can be optimized for the maximum catalyst productivity for a particular type of cycloaddition. The solvent can be also introduced as a catalyst carrier. The solvent can be introduced as a gas phase or as a liquid phase depending on the pressure and temperature. Advantageously, the solvent can be kept in the liquid phase and introduced as a liquid. Solvent can be introduced in the feed to the cycloaddition reactors.

The solution process can be performed in a batchwise fashion (e.g., batch; semi-batch) or in a continuous manner. Suitable reactors may include tank, loop, and tube designs. In at least one embodiment, the process is performed in a continuous fashion and dual loop reactors in a series configuration are used. In at least one embodiment, the process is performed in a continuous fashion and dual continuous stirred-tank reactors (CSTRs) in a series configuration are used. Furthermore, the process can be performed in a continuous fashion and a tube reactor can be used. In another embodiment, the process is performed in a continuous fashion and one loop reactor and one CSTR are used in a series configuration. The process can also be performed in a batchwise fashion and a single stirred tank reactor can be used.

Suitable diluents/solvents for the reaction may include non-coordinating, inert liquids. Examples of diluents/solvents include straight and branched-chain hydrocarbons, such as isobutane, butane, pentane, isopentane, hexanes, isohexane, heptane, octane, dodecane, and mixtures thereof; cyclic and acyclic hydrocarbons, such as cyclohexane, cycloheptane, methylcyclohexane, methylcycloheptane, and mixtures thereof, such as can be found commercially (Isopar™); halogenated hydrocarbons, such as perfluorinated C₄ to C₁₀ alkanes, chlorobenzene; and aromatic and alkylsubstituted aromatic compounds, such as benzene, toluene, mesitylene, and xylene. Suitable solvents may also include liquid olefins which may act as reactants including ethylene, propylene, 1-butene, 1-hexene, 1-pentene, 3-methyl-1-pentene, 4-methyl-1-pentene, 1-octene, 1-decene, 1,3-butadiene, isoprene, piperylene, myrcene, and mixtures thereof. In at least one embodiment, aliphatic hydrocarbon solvents are used as the solvent, such as isobutane, butane, pentane, isopentane, hexanes, isohexane, heptane, octane, dodecane, and mixtures thereof; cyclic and alicyclic hydrocarbons, such as cyclohexane, cycloheptane, methylcyclohexane, methylcycloheptane, and mixtures thereof. In an alternative embodiment, aromatic and alkylsubstituted aromatic compounds are used as the solvent, such as benzene, toluene, mesitylene, and xylene, or mixtures thereof.

The reactions can be run at any temperature and or pressure suitable to obtain the desired substituted-cyclobutanes, such as vinylcyclobutanes. Suitable temperatures and or pressures include a temperature of from about 0° C. to about 300° C., such as about 20° C. to about 200° C., such as about 35° C. to about 160° C., such as from about 35° C. to about 80° C., such as from about 50° C. to about 70° C. The reactions can be run at a pressure of from about 0.1 MPa to about 25 MPa, such as from about 0.45 MPa to about 6 MPa, or from about 0.5 MPa to about 4 MPa.

The run time of the reaction can be up to 1440 minutes, such as from about 60 minutes to 1440 minutes, such as from about 120 minutes to 1440 minutes, such as from about 180 minutes to 1440 minutes, such as from about 300 minutes to 1440 minutes.

In at least one embodiment, the reaction: 1) is conducted at temperatures of 0° C. to 300° C. (such as 20° C. to 200° C., such as 35° C. to 80° C., such as 50° C. to 70° C.); 2) is conducted at a pressure of atmospheric pressure to 10 MPa (such as 0.35 MPa to 10 MPa, such as from 0.45 MPa to 6 MPa, such as from 0.5 MPa to 4 MPa); 3) is conducted in an aliphatic hydrocarbon solvent (such as isobutane, butane, pentane, isopentane, hexanes, isohexane, heptane, octane, dodecane, and mixtures thereof; cyclic and alicyclic hydrocarbons, such as cyclohexane, cycloheptane, methylcyclohexane, methylcycloheptane, and mixtures thereof, such as can be found commercially (Isopar™)), aromatic and alkylsubstituted aromatic compounds (such as benzene, toluene, mesitylene, and xylene), or in the absence of solvent; 4) the catalyst used is [2,6-Bis(1-(2,6-dimethylphenylimino)pentyl)pyridine]iron butadiene 5) the reaction occurs in one reaction zone; and 6) the reactants are alpha-olefin and diene (such as ethylene and butadiene).

In at least one embodiment, the catalyst used in the reaction includes no more than one catalyst compound. A “reaction zone” is a vessel where the reaction takes place, for example a stirred-tank reactor or a loop reactor. When multiple reactors are used in a continuous process, each reactor is considered as a separate reaction zone. Room temperature is 23° C. unless otherwise noted.

### EXAMPLES

### General Considerations for Synthesis

All reagents were purchased from commercial vendors (Aldrich) and used as received unless otherwise noted. Solvents were sparged with N₂ and dried over 3 Å molecular sieves. Butadiene was filtered through basic alumina prior to use. All chemical manipulations were performed in a nitrogen environment unless otherwise stated. Flash column chromatography was carried out with Sigma Aldrich silica gel 60 Å (70 Mesh−230 Mesh) using solvent systems specified. NMR spectra were recorded on a Bruker 400 and/or 500 NMR with chemical shifts referenced to residual solvent peaks. All anhydrous solvents were purchased from Fisher Chemical and were degassed and dried over molecular sieves prior to use. Deuterated solvents were purchased from Cambridge Isotope Laboratories and were degassed and dried over molecular sieves prior to use. ¹H NMR spectroscopic data were acquired at 250 MHz, 400 MHz, or 500 MHz using solutions prepared by dissolving approximately 10 mg of a sample in either C₆D₆, CD₂Cl₂, CDCl₃, toluene-d₈, or other deuterated solvent. The chemical shifts (6) presented are relative to the residual protium in the deuterated solvent at 7.16 ppm, 5.32 ppm, 7.26 ppm, and 2.09 ppm for C₆D₆, CD₂Cl₂, CDCl₃, toluene-d₈, respectively.

Magnesium butadiene (also referred to as Mg(C₄H₆)·2THF or Mg(Butadiene)*THF₂ or Mg(butadiene)(THF)₂ or Mg(butadiene)(THF)₂) was prepared as described in US provisional patent application U.S. 63/154,043. [2,6-Bis(1-(2,6-dimethylphenylimino)ethyl)pyridine] iron dichloride was prepared as described in J. Am. Chem. Soc. 1998, 120 (16), pp. 4049-4050.

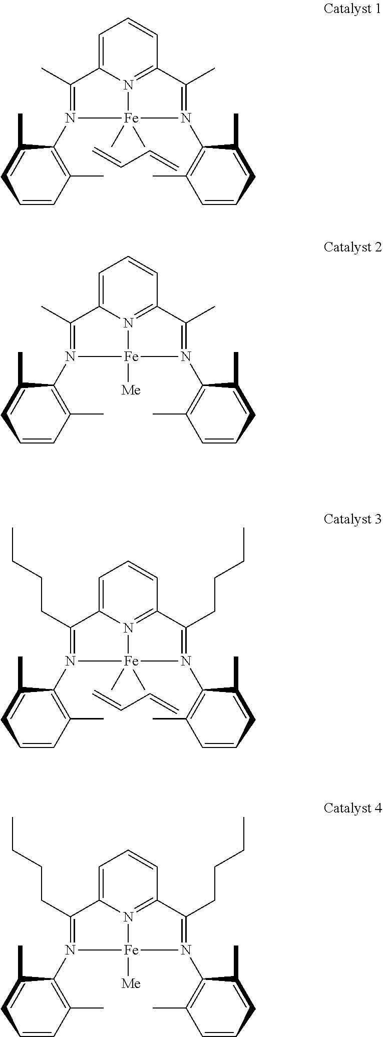

### Examples 1-9

### Synthesis of Ligands and Complexes (or Catalysts)

### Example 1: Preparation of [2,6-Bis(1-(2,6-dimethylphenylimino)ethyl)pyridine] iron butadiene (Catalyst 1)

[2,6-Bis(1-(2,6-dimethylphenylimino)ethyl)pyridine] iron dichloride (50 mg) was suspended in hexane (6 mL) and cooled to −30° C. Mg(butadiene)(THF)₂ was then added as a solid and the mixture was stirred at room temperature for 16 hours. The red mixture was filtered and the solvent was removed in vacuo to give a red powder (32 mg).

### Example 2: Preparation of [2,6-Bis(1-(2,6-dimethylphenylimino)ethyl)pyridine] iron methyl (Catalyst 2)

[2,6-Bis(1-(2,6-dimethylphenylimino)ethyl)pyridine] iron dichloride (1.20 g) was suspended in 30 mL diethyl ether and cooled to −30° C. A 1.6 M MeLi solution (3.02 mL) was then added and the mixture was stirred at room temperature for 10 minutes, after which it turned to deep green. The mixture was then filtered through Celite and the solvent was removed in vacuo to give a green powder (1.00 g).

### Example 3: Alternative Preparation of [2,6-Bis(1-(2,6-dimethylphenylimino)ethyl)pyridine] iron butadiene (Catalyst 1)

In a 20 mL vial, [2,6-Bis(1-(2,6-dimethylphenylimino)ethyl)pyridine] iron methyl (400 mg) in 10 mL benzene was frozen. Excess cold butadiene (1 mL) was added as a neat liquid and the capped solution was vigorously stirred at room temperature for 30 minutes. The cap of the vial was then removed and vigorous bubbling was observed, releasing the excess butadiene and ethane. Once the bubbling stopped, the solution was filtered through Celite. The resulting solution was subsequently used without further purification.

### Example 4: Preparation of [2,6-Bis(1-(2,6-dimethylphenylimino)pentyl)pyridine] iron dichloride

### 4.1 Preparation of 2,6-divaleroylpyridine

A solution of copper iodide (5.6 g, 29.4 mmol) was slurried into THF (50 mL) and cooled to −50° C. To this mixture, “BuLi in hexanes (2.48 M, 20 mL, 49 mmol) was slowly added in portions. The reaction mixture was stirred for an hour at −50° C. The mixture was then slowly added (over 100 minutes) to a chilled solution of the pyridine-2,6-dicarbonyl dichloride (5.0 g, 24.5 mmol) in THF (50 mL). The reaction mixture was stirred at ambient temperature over the weekend. To the dark brown reaction mixture, saturated ammonium chloride (2 mL) was added. The reaction was then concentrated to ˜40 mL, diluted with pentane (200 mL), and filtered. The filtride was washed with additional pentane (50 mL) and the filtrate was concentrated to a dark brown oil. The oil was then purified through a silica plug eluted with 50:50 diethyl ether and pentane to afford a light orange oil (5.08 g, 83.8%), which was used without further purification. ¹H NMR (400 MHz, CDCl₃) δ 8.18 (d, J=7.7 Hz, 2H), 7.97 (t, J=7.8 Hz, 1H), 3.25 (t, J=7.4 Hz, 4H), 1.75 (p, J=7.5 Hz, 4H), 1.45 (q, J=7.4 Hz, 4H), 0.97 (t, J=7.3 Hz, 6H).

### 4.2 Preparation of 2,6-Bis(1-(2,6-dimethylphenylimino)pentyl)pyridine

2,6-divaleroylpyridine (3.93 g, 15.9 mmol), 2,6-dimethylaniline (5.78 g, 47.7 mmol), and p-toluenesulfonic acid (151 mg, 0.8 mmol) were dissolved in toluene (50 mL). The reaction mixture was refluxed overnight using a Dean-Stark apparatus and then allowed to cool to ambient temperature. The toluene was removed by evaporation, and the solids were recrystallized from methanol to give a yellow solid (4.88 g, 67.6%). ¹H NMR (400 MHz, CDCl₃) δ 8.40 (d, J=7.8 Hz, 2H), 7.91 (t, J=7.8 Hz, 1H), 7.08 (d, J=7.5 Hz, 4H), 6.95 (t, J=7.5 Hz, 2H), 2.76-2.61 (m, 4H), 2.09 (s, 12H), 1.50-1.31 (m, 4H), 1.23 (h, J=7.3 Hz, 4H), 0.75 (t, J=7.3 Hz, 6H).

### 4.3 Preparation of [2,6-Bis(1-(2,6-dimethylphenylimino)pentyl)pyridine] iron dichloride

THF (30 mL) was added to 2,6-Bis(1-(2,6-dimethylphenylimino)pentyl)pyridine (4.88 g, 10.7 mmol) and iron dichloride (1.36 g, 10.7 mmol). The resulting mixture was stirred overnight at ambient temperature. The THF was removed by evaporation. The resulting solid was slurried into diethyl ether and isolated by filtration to give a blue solid (5.36 g, 85.9%). ¹H NMR (400 MHz, CD₂Cl₂) δ 84.71 (s, 2H), 42.40 (s, 1H), 16.14 (s, 4H), 12.06 (br, 12H), 7.41 (s, 4H), −0.63 (s, 4H), −1.51 (s, 6H), −4.01 (s, 4H), −11.91 (s, 2H).

### Example 5: Preparation of [2,6-Bis(1-(2,6-dimethylphenylimino)pentyl)pyridine] iron butadiene (Catalyst 3)

To a 20 mL vial of 2,6-Bis[1-(2,6-dimethylphenylimino)pentyl]pyridine iron dichloride (500 mg, 8.6 mmol) and Mg(C₄H₆)-2THF (192 mg, 8.6 mmol) at −20° C., pentane (ca. 6 mL) was added. To the cooled suspension at −20° C., diethyl ether (ca. 1 mL) was added dropwise. The reaction was subsequently stirred at ambient temperature for 80 minutes. The reaction was filtered through Celite on a glass fiber plug and the filtride was washed with a 1:1 solution of diethyl ether:pentane. Combined extracts were concentrated under vacuum to give a dark red-brown solid (409 mg, 84%). ¹H NMR (500 MHz, C₆D₆) δ 8.15 (d, J=7.7 Hz, 2H), 7.56 (t, J=7.7 Hz, 1H), 6.95-6.73 (m, 6H), 4.63 (s, 2H), 3.31 (d, J=11.5 Hz, 2H), 2.71 (s, 2H), 2.43 (m, 4H), 1.71 (s, 6H), 1.40 (m, 10H), 1.08 (h, J=7.5 Hz, 4H), 0.65 (t, J=7.4 Hz, 6H).

Solubility measurement: To Catalyst 3 (100 mg) was added pentane (10 g), and the resulting mixture was stirred for 3.5 hours. A portion of the resulting mixture was filtered through Celite to provide a clear, dark red solution with a weight of 3.8234 g. This solution was concentrated under vacuum to afford 32.6 mg of Catalyst 3 indicative of a 0.85 wt % solubility in pentane.

### Example 6: Preparation of [2,6-Bis(1-(2,6-dimethylphenylimino)pentyl)pyridine] iron methyl (Catalyst 4)

MeLi (8.27 mL, 1.60 M, 2.0 equiv) was added slowly via syringe to a cold mixture of [2,6-Bis(1-(2,6-dimethylphenylimino)pentyl)pyridine] iron dichloride complex (3.85 g) at −20° C. in 30 mL diethyl ether. The mixture was stirred at room temperature for 15 minutes and filtered through Celite. Solvent was removed in vacuo to give a green powder in quantitative yield.

### Example 7: Alternative Preparation of [2,6-Bis(1-(2,6-dimethylphenylimino)pentyl)pyridine] iron butadiene (Catalyst 3)

In a 20 mL vial, [2,6-Bis(1-(2,6-dimethylphenylimino)pentyl)pyridine] iron methyl complex (424 mg) was dissolved in 2 mL benzene and frozen at −35° C. Cold butadiene (83 mg) was added as a neat liquid and the capped solution was vigorously stirred at room temperature for 30 minutes. The cap of the vial was then removed and vigorous bubbling was observed, releasing the excess butadiene and ethane. Once the bubbling stopped, the solution was filtered through Celite. The resulting solution was subsequently used without further purification.

### Example 8: Alternative Preparation of [2,6-Bis(1-(2,6-dimethylphenylimino)pentyl)pyridine] iron butadiene (Catalyst 3)

[2,6-Bis(1-(2,6-dimethylphenylimino)pentyl)pyridine] iron methyl complex (1.27 g) was dissolved in 10 mL pentane and cooled to −30° C. Cold butadiene was added as a neat liquid (261 mg) and the solution was stirred for 15 minutes at room temperature. The solution was then filtered through Celite and the solvent was removed in vacuo to yield 1.1 g of a red powder.

### Example 9: Alternative Preparation of [2,6-Bis(1-(2,6-dimethylphenylimino) pentyl)pyridine] iron butadiene (Catalyst 3)

[2,6-Bis(1-(2,6-dimethylphenylimino)pentyl)pyridine] iron methyl complex (4.50 g) was dissolved in 30 mL diethyl ether and cooled to −30° C. Cold excess butadiene (5 mL) was added as neat liquid and the solution was stirred at room temperature for 15 minutes. The solution was then filtered through Celite and the solvent was removed in vacuo to give 1.9 g of a red powder.

### Examples 10-14: Cycloaddition Reactions

1,3-Butadiene (99%) (Airgas) was further purified by passage through a 500 cc column packed with basic alumina (Aldrich Chemical Company, Brockman Basic 1).

Polymerization grade ethylene was further purified by passage through a 500 cm³ column packed with dried 5 Å molecular sieves (8 mesh-12 mesh; Aldrich Chemical Company).

Liquid samples were analyzed by gas chromatography with flame ionization detection (GC-FID) using an Agilent GC (7890), using the process described in Table 1. Select components and corresponding retention times were identified by comparison to an authentic compound and/or by analysis by gas chromatography-mass spectrometry (GC-MS), using the process described in Table 1. Composition of GC samples were assumed to be representative of the corresponding bulk sample, without further corrections for any loss of volatile components during transfer.

TABLE 1
GC Process

| Inlet Temperature | 200° | C. |
|---|---|---|
| Detector Temperature | 275° | C. |
| Sample Temp. Program | Initial 40° C. hold for 5 min | |
| | Ramp @15° C./min to 280° C., hold for 5 min | |
| Column Flow | 1 | mL/min |
| Split | Split mode; | |
| | 50:1 (sample diluted with CH₂Cl₂, ~1:5 v/v) | |
| | or 100:1 (neat sample) | |
| Injector | Autosampler (0.1 μL) | |
| Column | HP-PONA 19091S-001 Capillary Column; | |
| | 50 m × 0.2 mm × 0.5 μm | |
| | (100% dimethylpolysiloxane) | |
| H₂ Flow | 40 | mL/min |
| Air Flow | 400 | mL/min |
| Column Flow (He) | 10 | mL/min |

Samples were prepared either as a neat sample or by diluting approximately 200 μL of the analyte with dichloromethane (1 mL). The Figure illustrates a typical chromatogram of a sample containing vinylcyclobutane (VCB), butadiene, traces of cis-1,4-hexadiene (cis-1,4-1HD), and other components, diluted with dichloromethane.

Observed retention times for select components are listed in Table 2. Response factors for all observed peaks, corresponding to identified or unidentified components, were assumed to be equal to unity (area %=wt %), with the exception of any peak corresponding to dichloromethane (area % dichloromethane=0 wt %). The ratio of vinylcyclobutane to cis-1,4-hexadiene was calculated using the following formula:

$$ratio of VCB to 1 , 4 ‐ HD = \frac{wt \% VCB}{wt \% cis ‐ 1 , 4 ‐ HD}$$

TABLE 2
Observed retention times for select components

Retention Time Range

| Component | From | To |
|---|---|---|
| 1,3-Butadiene | 3.769 | 3.807 |
| Dichloromethane (diluent) | 5.232 | 5.324 |
| 1,5-Hexadiene | 6.499 | 6.532 |
| Trans-1,4-Hexadiene | 6.939 | 6.984 |
| Cis-1,4-Hexadiene | 7.137 | 7.173 |
| Vinylcyclobutane | 7.481 | 7.738 |
| 2,4-Hexadiene isomer | 8.242 | 8.284 |
| Benzene (solvent) | 8.286 | 8.328 |
| 2,4-Hexadiene isomer | 8.509 | 8.552 |
| Ethylidene cyclobutane (assumed) | 8.639 | 8.682 |
| 2,4-Hexadiene isomer | 8.719 | 8.763 |
| Cyclohexene | 8.875 | 8.919 |
| Toluene (solvent) | 10.554 | 10.700 |
| 4-Vinylcyclohexene | 11.899 | 11.958 |
| 1,5-Cyclooctadiene | 13.396 | 13.464 |

### Example 10: Cycloaddition of Butadiene with Ethylene Using Catalyst 3, as Prepared in Example 7

In a glovebox, the catalyst solution prepared in Example 7 was loaded into a stainless steel injection cylinder which was attached to a 2 L autoclave reactor. To the 2 L autoclave reactor was injected liquid butadiene (300 mL) under a nitrogen atmosphere. The impeller was allowed to stir at 450 rpm. The reactor was heated to 70° C., and ethylene (350 psig) was added. The reactor was then heated to 75° C., and the catalyst mixture was injected with ethylene (15 psid), leading to a total ethylene partial pressure of 365 psig. The reactor was then heated to 80° C. for 5 hours under a steady-state ethylene pressure of 365 psig. The reactor was then cooled to ambient temperature, vented, and opened.

The resulting liquid was filtered through basic alumina to remove catalyst residue. GC analysis of the filtrate (221 g) indicated 82.57 wt % vinylcyclobutane and 1.33 wt % cis-1,4-hexadiene, for a vinylcyclobutane to cis-1,4-hexadiene ratio of 62.1 to 1.

### Example 10: Cycloaddition of butadiene with ethylene using Catalyst 3, as prepared in Example 8

In a glovebox, [2,6-bis(1-(2,6-dimethylphenylimino)pentyl)pyridine] iron butadiene (416 mg) was dissolved in toluene (8 mL). The resulting catalyst solution was loaded into a stainless steel injection cylinder which was attached to a 2 L autoclave reactor. To the 2 L autoclave reactor was injected liquid butadiene (300 mL) under a nitrogen atmosphere. The impeller was allowed to stir at 450 rpm. The reactor was heated to 50° C., and ethylene (300 psig) was added. The reactor was then heated to 55° C., and the catalyst mixture was injected with ethylene (100 psid), leading to a total ethylene partial pressure of 400 psig. The reactor was then heated to 60° C. for 5 hours under a steady-state ethylene pressure of 400 psig. The reactor was then cooled to ambient temperature, vented, and opened. The resulting liquid was filtered through silica to remove catalyst residue. GC analysis of the filtrate (242 g) indicated 83.02 wt % vinylcyclobutane and 0.49 wt % cis-1,4-hexadiene, for a vinylcyclobutane to cis-1,4-hexadiene ratio of 169 to 1.

A second batch of vinylcyclobutane was produced in a similar fashion. In a glovebox, [2,6-bis(1-(2,6-dimethylphenylimino)pentyl)pyridine] iron butadiene (600 mg) was dissolved in toluene (12 mL). The resulting catalyst solution was loaded into a stainless steel injection cylinder which was attached to a 2 L autoclave reactor. To the 2 L autoclave reactor was injected liquid butadiene (400 mL) under a nitrogen atmosphere. The impeller was allowed to stir at 450 rpm. The reactor was heated to 50° C., and ethylene (300 psig) was added. The reactor was then heated to 55° C., and the catalyst mixture was injected with ethylene (100 psid), leading to a total ethylene partial pressure of 400 psig. The reactor was then heated to 60° C. for 75 minutes under a steady-state ethylene pressure of 400 psig. The reactor was then cooled to ambient temperature, vented, and opened. The resulting liquid was filtered through silica to remove catalyst residue. GC analysis of the filtrate (108 g) indicated 61.30 wt % vinylcyclobutane and 0.26 wt % cis-1,4-hexadiene, for a vinylcyclobutane to cis-1,4-hexadiene ratio of 236 to 1.

The filtrate from the two reactions were combined and purified by distillation from sodium-potassium alloy under nitrogen at 1 atm (vinylcyclobutane b.p. ˜ 68-70° C.) to afford 229 g of vinylcyclobutane in 98.7 wt % purity (0.5 wt % cis-1,4-hexadiene).

### Example 11: Cycloaddition of Butadiene with Ethylene Using Catalyst 3, as Prepared in Example 9

In a glovebox, [2,6-bis(1-(2,6-dimethylphenylimino)pentyl)pyridine] iron butadiene (100 mg) was dissolved in toluene (10 mL). The resulting catalyst solution was loaded into a stainless steel injection cylinder which was attached to a 2 L autoclave reactor. To the 2 L autoclave reactor was injected liquid butadiene (300 mL) under a nitrogen atmosphere. The impeller was allowed to stir at 450 rpm. The reactor was heated to 50° C., and ethylene (300 psig) was added. The reactor was then heated to 55° C., and the catalyst mixture was injected with ethylene (100 psid), leading to a total ethylene partial pressure of 400 psig. The reactor was then heated to 60° C. for 7.3 hours under a steady-state ethylene pressure of 400 psig, then for 15.5 hours isolated, and finally for 1.2 hours under a steady-state ethylene pressure of 400 psig. The reactor was then cooled to ambient temperature, vented, and opened. The resulting liquid was filtered through silica to remove catalyst residue. GC analysis of the filtrate (186 g- filtered) indicated 80.34 wt % vinylcyclobutane and 0.21 wt % cis-1,4-hexadiene, for a vinylcyclobutane to cis-1,4-hexadiene ratio of 380 to 1.

A second batch of vinylcyclobutane was produced in a similar fashion. In a glovebox, [2,6-bis(1-(2,6-dimethylphenylimino)pentyl)pyridine] iron butadiene (300 mg) was dissolved in toluene (10 mL). The resulting catalyst solution was loaded into a stainless steel injection cylinder which was attached to a 2 L autoclave reactor. To the 2 L autoclave reactor was injected liquid butadiene (300 mL) under a nitrogen atmosphere. The impeller was allowed to stir at 450 rpm. The reactor was heated to 50° C., and ethylene (300 psig) was added. The reactor was then heated to 55° C., and the catalyst mixture was injected with ethylene (100 psid), leading to a total ethylene partial pressure of 400 psig. The reactor was then heated to 60° C. for 6.8 hours under a steady-state ethylene pressure of 400 psig, then for 16 hours isolated, and finally for 1.2 hours under a steady-state ethylene pressure of 400 psig. The reactor was then cooled to ambient temperature, vented, and opened. The resulting liquid was filtered through silica to remove catalyst residue. GC analysis of the filtrate (483 g) indicated 61.30 wt % vinylcyclobutane and 0.26 wt % cis-1,4-hexadiene, for a vinylcyclobutane to cis-1,4-hexadiene ratio of 236 to 1.

The filtrate from the two reactions were combined and purified by distillation from sodium-potassium alloy under nitrogen at 1 atm (vinylcyclobutane b.p. ˜ 70° C.) to afford 357 g of vinylcyclobutane in 99.7 wt % purity (0.2 wt % cis-1,4-hexadiene).

### Example 12: Cycloaddition of butadiene with butadiene using Catalyst 4 prepared from [2,6-Bis(1-(2,6-dimethylphenylimino)pentyl)pyridine] Iron Dichloride Complex

[2,6-Bis(1-(2,6-dimethylphenylimino)pentyl)pyridine] iron dichloride complex (100 mg) in 1 mL diethyl ether was cooled to −30° C. and methyllithium in diethyl ether (1.6 M, 215 μL, 2 equiv.) was added. The solution was then filtered through Celite into a thick walled flask where it was combined with excess butadiene (˜1 mL). The solution was then stirred at 50° C. for 16 h. The yellow solids that precipitated from the solution was collected by filtration, washed with dichloromethane, chloroform and ethyl acetate, and dried in vacuo. NMR analysis of the solids indicated a number-average molecular mass (M.) of 583 g mol⁻¹, corresponding to an average of ten cyclobutyl repeat units.

### Example 13: Cycloaddition of Butadiene with Butadiene Using Catalyst 3

[2,6-Bis(1-(2,6-dimethylphenylimino)pentyl)pyridine] iron butadiene complex (20 mg) was dissolved in 3 mL diethyl ether and placed in a thick walled glass reaction vessel. The solution as then cooled in the freezer to −35° C. Butadiene (˜2 g) was added and the flask was stirred at 50° C. for 16 h. The flask was opened and excess butadiene was released. The solution was then filtered using hexanes. The solvent from filtrate was removed to give −350 mg of a yellow powder.

### Comparative Example 1: Cycloaddition of butadiene with ethylene using Catalyst 1, as prepared in Example 3

In a glovebox, the catalyst solution prepared in Example 3 was loaded into a stainless steel injection cylinder which was attached to a 2 L autoclave reactor. To the 2 L autoclave reactor was injected liquid butadiene (300 mL) under a nitrogen atmosphere. The impeller was allowed to stir at 450 rpm. The reactor was heated to 70° C., and ethylene (350 psig) was added. The reactor was then heated to 75° C., and the catalyst mixture was injected with ethylene (50 psid), leading to a total ethylene partial pressure of 400 psig. The reactor was then heated to 80° C. for 3 hours under a steady-state ethylene pressure of 400 psig. The reactor was then cooled to ambient temperature, vented, and opened.

The resulting liquid was filtered through basic alumina to remove catalyst residue. GC analysis of the filtrate (230 g) indicated 77.66 wt % vinylcyclobutane and 2.13 wt % cis-1,4-hexadiene, for a vinylcyclobutane to cis-1,4-hexadiene ratio of 36.5 to 1.

As can be seen in Examples 10-12, the inventive Catalyst 3 produces a higher ratio vinycyclobutane (product) to cis-1,4-hexadiene (side product).

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the present disclosure, additionally, they do not exclude impurities and variances normally associated with the elements and materials used.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby. Likewise, the term “comprising” is considered synonymous with the term “including” for purposes of United States law. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

While the present disclosure has been described with respect to a number of embodiments and examples, those skilled in the art, having benefit of this disclosure, will appreciate that other embodiments can be devised which do not depart from the scope and spirit of the present disclosure.

What is claimed is:

1. A catalyst compound represented by Formula (I) or Formula (II): 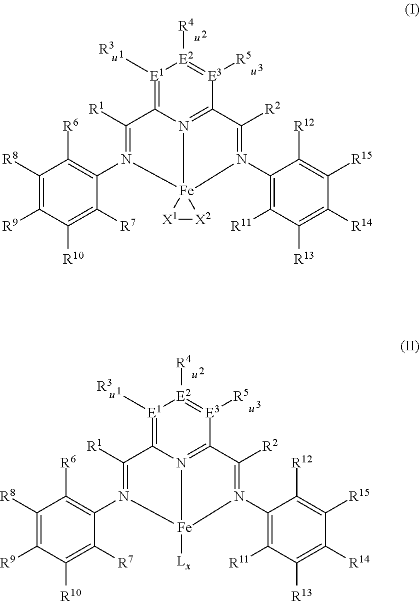
wherein:
each of R¹ and R² is independently a C₄-C₂₂ linear, branched or cyclic alkyl or alkenyl, wherein each of R¹ and R² is optionally substituted by halogen, —OR¹⁶, —NR¹⁷₂, or —SiR¹⁸₃;
each of X¹ and X² is independently a C₁ to C₂₀ hydrocarbyl where X¹ and X² form a diene or an alkenyl diradical, optionally substituted by halogen or —SiR¹⁸₃;
each of R³, R⁴, R⁵, R⁸, R⁹, R¹⁰, R¹³, R¹⁴, and R¹⁵ is independently hydrogen, C₁-C₂₂ alkyl, C₂-C₂₂ alkenyl, C₆-C₂₂ aryl, C₇-C₃₀ arylalkyl, —OR¹⁶, —NR¹⁷₂, halogen, —SiR¹⁸₃ or five-, six- or seven-membered heterocyclic ring comprising at least one atom selected from the group consisting of N, P, O and S;
wherein R³, R⁴, R⁵, R⁸, R⁹, R¹⁰, R¹³, R¹⁴, and R¹⁵ are optionally substituted by halogen, —OR¹⁶, —NR¹⁷₂, or —SiR¹⁸₃; wherein R³ optionally bonds with R⁴, R⁴ optionally bonds with R⁵, R⁷ optionally bonds with R¹⁰, R¹⁰ optionally bonds with R⁹, R⁹ optionally bonds with R⁸, R⁸ optionally bonds with R⁶, R¹² optionally bonds with R¹⁵, R¹⁵ optionally bonds with R¹⁴, R¹⁴ optionally bonds with R¹³, and R¹³ optionally bonds with R¹¹, in each case to independently form a five-, six- or seven-membered carbocyclic or heterocyclic ring, the heterocyclic ring comprising at least one atom from the group consisting of N, P, O and S;
each of R⁶, R⁷, R¹¹, and R¹² is independently hydrogen, C₁-C₂₂ alkyl, C₂-C₂₂ alkenyl, C₆-C₂₂ aryl, C₇-C₃₀ arylalkyl, a heteroatom or a heteroatom-containing group, or five-, six- or seven-membered heterocyclic ring comprising at least one atom selected from the group consisting of N, P, O and S; wherein R⁶, R⁷, R¹¹, and R¹² are optionally substituted by halogen, —OR¹⁶, —NR¹⁷₂, or —SiR¹⁸₃, wherein R⁶ optionally bonds with R⁸, R⁷ optionally bonds with R¹⁰, R¹¹ optionally bonds with R¹³, or R¹⁵ optionally bonds with R¹² in each case to independently form a five-, six-or seven-membered carbocyclic or heterocyclic ring, the heterocyclic ring comprising at least one atom from the group consisting of N, P, O and S;
each of R¹⁶, R¹⁷, and R¹⁸ is independently hydrogen, C₁-C₂₂ alkyl, C₂-C₂₂ alkenyl, C₆-C₂₂ aryl, C₇-C₃₀ arylalkyl, or —SiR¹⁹₃, wherein each R¹⁶, R¹⁷, and R¹⁸ is independently optionally substituted by halogen, or two R¹⁶ radicals optionally bond to form a five- or six-membered ring, or two R¹⁷ radicals optionally bond to form a five- or six-membered ring, or two R¹⁸ radicals optionally bond to form a five- or six-membered ring;
each R¹⁹ is independently hydrogen, C₁-C₂₂ alkyl, C₂-C₂₂ alkenyl, C₆-C₂₂ aryl, C₇-C₃₀ arylalkyl, or two R¹⁹ radicals optionally bond to form a five- or six-membered ring;
each of E¹, E², and E³ is independently carbon, nitrogen or phosphorus;
each of u¹, u², and u³ indicates the presence of R³, R⁴ and R⁵, respectively, and is 0 when E¹, E², or E³ is nitrogen or phosphorus, and is 1 when E¹, E², or E³ is carbon;
L is either a dinitrogen group when x is 2 and a single L is optionally bonded to a second metal center, or L is a methyl group when x is 1.

2. The catalyst of claim 1, wherein each of E¹, E², and E³ is carbon, and R³, R⁴, and R⁵ are independently hydrogen or C₁-C₂₂ alkyl.

3. The catalyst of claim 2, wherein R³, R⁴, and R⁵ is hydrogen.

4. The catalyst of claim 1, wherein X¹ and X² form a C4-C32 conjugated diene or alkenyl diradical, optionally substituted by halogen or —SiR¹⁸₃.

5. The catalyst of claim 1, wherein X¹ and X² form 1,3-butadiene, 1,3-pentadiene, isoprene, myrcene, or 2-hexene-1,6-diyl.

6. The catalyst of claim 1, wherein R⁶, R⁷, R¹¹, and R¹² are independently selected from methyl, ethyl, propyl, butyl, pentyl, hexyl, heptyl, octyl, nonyl, decyl, undecyl, dodecyl, cyclopentyl, phenyl, substituted phenyl, biphenyl or an isomer thereof, substituted hydrocarbyl radicals and all isomers of substituted hydrocarbyl radicals comprising trimethylsilylpropyl, trimethylsilylmethyl, trimethylsilylethyl, phenyl, or all isomers of hydrocarbyl substituted phenyl including methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, or dipropylmethylphenyl, or isomers thereof.

7. The catalyst of claim 1, wherein each of R¹ and R² is independently C₄-C₁₂ alkyl.

8. The catalyst of claim 1, wherein each of R¹ and R² is selected from butyl, pentyl, hexyl, heptyl, octyl, nonyl, decyl, undecyl, dodecyl or an isomer thereof.

9. The catalyst of claim 1, wherein R¹ is butyl and R² is butyl.

10. The catalyst of claim 1, wherein the catalyst compound is selected from:
[2,6-Bis(1-(2,6-dimethylphenylimino)pentyl)pyridine] iron butadiene;
[2,6-Bis(1-(2,6-dimethylphenylimino)pentyl)pyridine] iron isoprene;
[2,6-Bis(1-(2,6-dimethylphenylimino)pentyl)pyridine] iron myrcene;
[2,6-Bis(1-(2,6-dimethylphenylimino)pentyl)pyridine] iron methyl;
[2,6-Bis(1-(2,6-dimethylphenylimino)pentyl)pyridine] iron 2-hexene-1,6-diyl;
[2,6-Bis(1-(2,6-dimethylphenylimino)hexyl)pyridine] iron butadiene;
[2,6-Bis(1-(2,6-dimethylphenylimino)hexyl)pyridine] iron isoprene;
[2,6-Bis(1-(2,6-dimethylphenylimino)hexyl)pyridine] iron myrcene;
[2,6-Bis(1-(2,6-dimethylphenylimino)hexyl)pyridine] iron 2-hexene-1,6-diyl;
[2,6-Bis(1-(2,6-dimethylphenylimino)heptyl)pyridine] iron butadiene;
[2,6-Bis(1-(2,6-dimethylphenylimino)heptyl)pyridine] iron isoprene;
[2,6-Bis(1-(2,6-dimethylphenylimino)heptyl)pyridine] iron myrcene;
[2,6-Bis(1-(2,6-dimethylphenylimino)heptyl)pyridine] iron 2-hexene-1,6-diyl;
[2,6-Bis(1-(2,6-dimethylphenylimino)octyl)pyridine] iron butadiene;
[2,6-Bis(1-(2,6-dimethylphenylimino)octyl)pyridine] iron isoprene;
[2,6-Bis(1-(2,6-dimethylphenylimino)octyl)pyridine] iron myrcene;
[2,6-Bis(1-(2,6-dimethylphenylimino)octyl)pyridine] iron 2-hexene-1,6-diyl;
[2,6-Bis(1-(2,6-dimethylphenylimino)nonyl)pyridine] iron butadiene;
[2,6-Bis(1-(2,6-dimethylphenylimino)nonyl)pyridine] iron isoprene;
[2,6-Bis(1-(2,6-dimethylphenylimino)nonyl)pyridine] iron myrcene; and
[2,6-Bis(1-(2,6-dimethylphenylimino)nonyl)pyridine] iron 2-hexene-1,6-diyl.

11. The catalyst of claim 1, wherein the catalyst is generated by addition of a conjugated diene or vinylcyclobutane to the following complex in a hydrocarbon solvent:

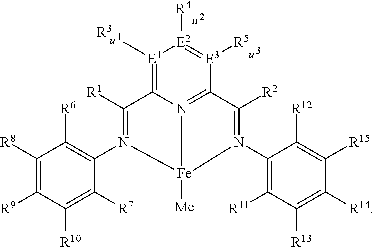

12. The catalyst of claim 11, wherein the catalyst is purified by filtration to remove any insoluble impurities.

13. A process for making substituted-cyclobutanes, comprising:
reacting a first olefin and a second olefin in the presence of the catalyst of claim 1 at a reaction pressure of from 0.05 MPa to 1,500 MPa and a reaction temperature of from 30° C. to 230° C. to form the substituted-cyclobutane.

14. The process of claim 13, wherein the first olefin is selected from C4 to C32 conjugated dienes and the second olefin is selected from C2 to C32 alpha olefins.

15. The process of claim 13, wherein the substituted-cyclobutane is vinylcyclobutane and the first olefin is butadiene and the second olefin is ethylene.

16. The process of claim 13, wherein the first olefin is selected from C4 to C32 conjugated dienes and the second olefin is selected from C4 to C32 dienes.

17. The process of claim 13, wherein the first olefin is selected from C4 to C32 conjugated dienes and the second olefin is selected from C4 to C32 conjugated dienes.

18. The process of claim 13, wherein the first olefin is butadiene and the second olefin is butadiene.

19. The process of claim 13, wherein the first olefin is selected from C3 to C32 alpha olefins, the second olefin is selected from C3 to C32 alpha olefins, and the first olefin and the second olefin may be the same or different.

20. The process of claim 13, wherein the first olefin is selected from C4 to C32 dienes and the second olefin and is selected from C4 to C32 dienes, and the first olefin and the second olefin may be the same or different.

21. The process of claim 20, wherein the first olefin and the second olefin are butadiene.

22. A process for making substituted-cyclobutane, comprising:
reacting a first olefin and a second olefin in the presence of the catalyst of claim 1 to provide a reaction product comprising the substituted-cyclobutane.

23. The process of claim 22, wherein the substituted-cyclobutane is vinylcyclobutane.

24. The process of claim 23, wherein the reaction product further comprises cis-1,4-hexadiene and wherein a ratio of vinylcyclobutane to cis-1,4-hexadiene produced is equal to or greater than 49 to 1, as measured by GC-FID.
